(19) United States
(12) Patent Application Publication
(10) Pub. No.: US 2022/0151553 A1
(43) Pub. Date: May 19, 2022

(54) SMARTWATCH WITH NON-INVASIVE ANALYTE SENSOR

(71) Applicant: Know Labs, Inc., Seattle, WA (US)

(72) Inventor: Phillip BOSUA, Seattle, WA (US)

(21) Appl. No.: 17/455,309

(22) Filed: Nov. 17, 2021

Related U.S. Application Data
(60) Provisional application No. 63/115,178, filed on Nov. 18, 2020.

(51) Int. Cl. A61B 5/00; A61B 5/145; A61B 5/0205

(52) U.S. Cl. CPC A61B 5/681; A61B 5/0205; A61B 5/14532; A61B 5/0075

(57) ABSTRACT

A smartwatch includes a non-invasive analyte sensor that is configured to non-invasively detect an analyte in a person wearing the smartwatch via spectroscopic techniques using non-optical frequencies such as in the radio or microwave frequency bands of the electromagnetic spectrum. The smartwatch can also include other functionality such as time keeping, date keeping, one or more additional sensors configured to sense one or more physiological properties of the wearer. Data generated by the analyte sensor and other data collected by the smartwatch or by a sensing device separate from the smartwatch can be analyzed to look for correlations between the analyte data and the other data. The results of the analysis can then be sent to the smartwatch to advise the user of the results.

### FIELD

This technical disclosure relates to a smartwatch that includes a non-invasive analyte sensor in addition to other smartwatch functionality.

### BACKGROUND

A sensor that uses radio or microwave frequency bands of the electromagnetic spectrum for in vivo medical diagnostics is disclosed in U.S. Pat. No. 10,548,503. Additional examples of sensors that purport to be able to use radio or microwave frequency bands of the electromagnetic spectrum to detect a substance in a person are disclosed in U.S. Patent Application Publication 2019/0008422 and U.S. Patent Application Publication 2020/0187791.

### SUMMARY

A smartwatch and a system that uses the smartwatch are described. The smartwatch includes a non-invasive analyte sensor that is configured to non-invasively detect an analyte in a person wearing the smartwatch via spectroscopic techniques using non-optical frequencies such as in the radio or microwave frequency bands of the electromagnetic spectrum. The smartwatch also includes other functionality such as time keeping, date keeping, one or more additional sensors configured to sense one or more physiological properties of the wearer.

In some embodiments, data from the analyte sensor and data from the other functionality of the smartwatch can be collected and analyzed. In additional embodiments, the other/additional data can be collected using a second sensor, which may be a wearable sensor or a non-wearable sensor. Using the collected data, one or more correlations between the analyte data and the other data can be determined. The correlation(s) can aid a caregiver, such as a doctor or other caregiver, in a diagnoses of a medical condition of the wearer and/or determine a treatment plan for the wearer, and/or the correlations can provide information to the wearer in regulating their physiological condition, and/or the correlation(s) can be used for long term tracking of a physiological condition of the wearer.

The analyte sensor of the smartwatch can be used to detect the presence of the analyte of interest, as well an amount of the analyte or a concentration of the analyte within the wearer of the smartwatch. The analyte can include, but is not limited to, one or more of blood glucose, blood alcohol, white blood cells, or luteinizing hormone.

In addition to the non-invasive analyte sensor, the smartwatch can also include one or more additional sensors configured to sense one or more physiological properties of the wearer. Example of a physiological property that can be sensed include, but are not limited to, user temperature; user heart rate; user blood pressure; user oxygen saturation; or a bioelectric impedance. The smartwatch can also include one or more additional functionalities including, but not limited to, a camera; an accelerometer; a pedometer; a fitness/activity tracker; an altimeter; a barometer; a compass; a global positioning system; a sleep monitor; a fall sensor; a microphone; and a speaker.

In another embodiment, the one or more additional sensors that are configured to sense the one or more physiological properties of the wearer can be in a second wearable or non-wearable sensor separate from the smartwatch.

In one embodiment described herein, a smartwatch can include a housing portion having a touchscreen display on an upper face thereof, at least one rechargeable battery within the housing portion providing electrical power, and a non-invasive analyte sensor at least partially in the housing portion. The non-invasive analyte sensor can include at least one transmit antenna and at least one receive antenna, where the at least one transmit antenna is positioned and arranged to transmit a signal into a person wearing the smartwatch, and the signal is in a radio or microwave frequency range of the electromagnetic spectrum. The at least one receive antenna is positioned and arranged to detect a response resulting from transmission of the signal by the at least one transmit antenna into the person. The smartwatch further includes a first stored mobile application that interfaces with and controls operation of the non-invasive analyte sensor and whereby an analyte measurement obtained by the non-invasive analyte sensor can be displayed on the touchscreen display, a time-keeping circuit whereby a current time can be displayed on the touchscreen display, a date-keeping circuit whereby a current date can be displayed on the touchscreen display, and a wristband attached to the housing portion.

In another embodiment, a system can include the smartwatch described herein, and a data analysis system in electronic communication with and receiving data collected by the smartwatch. The data includes first data on an analyte detected by the non-invasive analyte sensor and second data collected by the smartwatch. In another embodiment, the second data can be collected by a second wearable or non-wearable sensor separate from the smartwatch. The data analysis system is programmed to determine one or more correlations between the first data and the second data. The data analysis system can be included in the smartwatch or it can be separate from the smartwatch.

### DRAWINGS

FIG. 1 is a perspective view of a portion of a smartwatch described herein.

FIG. 2 is a schematic depiction of the smartwatch of FIG. 1 showing some components of the smartwatch.

FIG. 3 is a schematic side view of the smartwatch relative to a person's arm when the smartwatch is worn showing operation of the non-invasive analyte sensor.

FIG. 4A is a schematic view of a system that can include the smartwatch described herein.

FIG. 4B is a schematic view of another embodiment of a system that can include the smartwatch described herein.

FIG. 5A illustrates an embodiment of a method described herein.

FIG. 5B illustrates another embodiment of a method described herein.

FIG. 6 illustrates an embodiment of an interchangeable smartwatch system.

### DETAILED DESCRIPTION

The following is a detailed description of a smartwatch, a system that employs the smartwatch, and methods involving use of data collected by the smartwatch. A smartwatch as used herein refers to a digital watch that is worn around a user's wrist/arm, has a touchscreen display that allows the user to navigate watch functionality and perform actions by touching or hovering over the touchscreen. Examples of conventional smartwatches include, but are not limited to, the Apple Watch™, the Samsung Galaxy™ Watch, and the Fitbit™, and many others. The non-invasive analyte sensor described herein can be incorporated into any type of smartwatch.

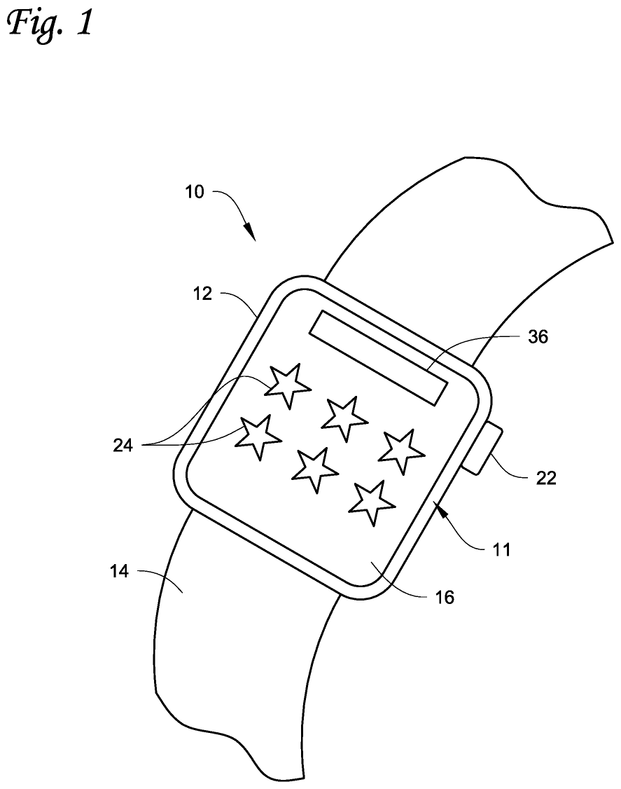

With reference to FIG. 1, a smartwatch 10 that incorporates the concepts described herein is illustrated. The smartwatch 10 includes a sensing device 11 having a housing portion 12 that houses the electronics and other components of the smartwatch 10. A wristband 14 is attached to the housing portion 12 for removably securing the smartwatch 10 around a user's wrist.

Referring to FIGS. 1-3, a touchscreen display 16 is disposed at an upper side of the housing portion 12 and forms a portion of an upper face thereof. The housing portion 12 includes a lower side 18 that faces the user's arm 20 or lower arm, particularly the user's wrist, when the smartwatch 10 is worn. A function/selection/navigation button 22 (seen in FIG. 1) is disposed on a side of the housing portion 12 which may be used to launch a function of the smartwatch, select a function, and/or navigate through options displayed on the touchscreen display 16.

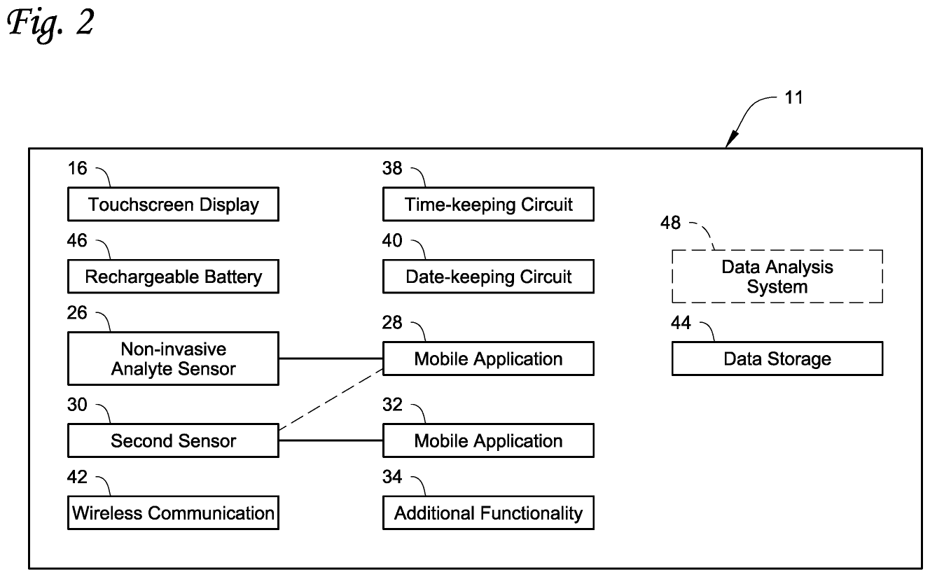

The smartwatch 10 displays various icons 24 on the touchscreen display 16. Each icon 24 represents an App (i.e. a mobile application) that can be launched (i.e. initiated) by selecting the icon 24, for example using either the user's finger or using the button 22. Each App controls a functionality of the smartwatch 10. For example, as described in further detail below, the smartwatch 10 can include a non-invasive analyte sensor 26 along with an App 28 (best seen in FIG. 2) that initiates the operation of and controls the operation of the non-invasive analyte sensor 26. One of the icons 24 will be associated with the App 28 to launch the App 28 and start and optionally stop operation of the sensor 26.

The display 16 can also display information relating to the sensor 26, such as information on the sensor readings and/or information relating to the analysis of the data collected by the sensor 26. For example, the display 16 can display charts, number-based readings, data analysis outcomes, and other information.

The smartwatch 10 can also include at least one second or additional sensor 30 along with an associated App 32 (best seen in FIG. 2) that initiates the operation of and controls the operation of the second sensor 30. In another embodiment best seen in FIG. 2, the App 28 can control both the analyte sensor 26 and the second sensor 30. One of the icons 24 will be associated with the App 32 to launch the App 32 and start and optionally stop operation of the sensor 30. The sensor 30 can be configured to sense one or more physiological properties of the user. Examples of the sensor 30 that can be used include, but are not limited to, a user temperature sensor such as a thermal scanner or a thermometer; a heart rate monitor for detecting the heart rate of the user; a blood pressure monitor that detects the blood pressure of the user; an oxygen saturation monitor that detects oxygen saturation of the user, or a bioelectric impedance monitor that takes electrical impedance readings of the user. Other sensors are possible.

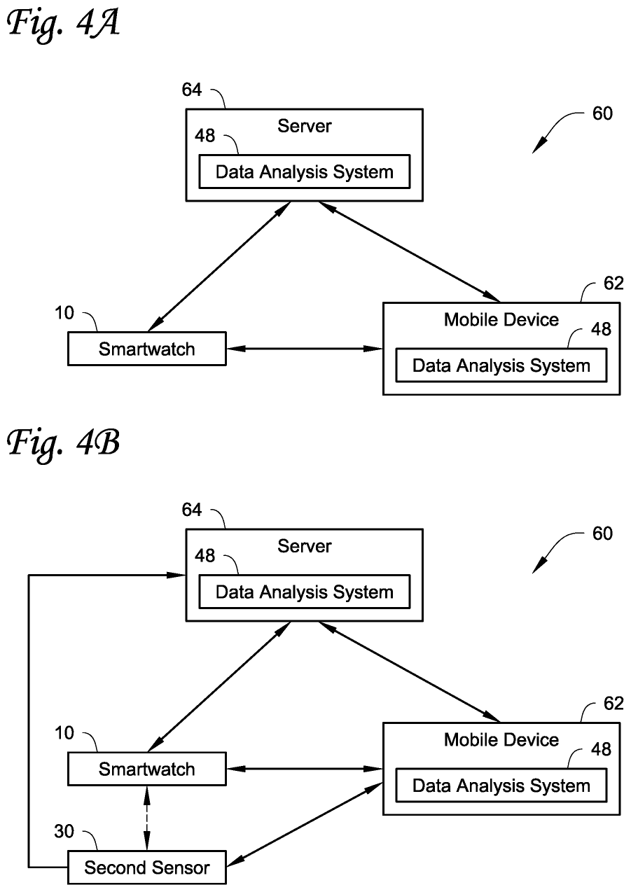

Instead of the second sensor 30 being on the smartwatch 10, the second sensor 30 can be part of a sensing device that is separate from the smartwatch 10 as depicted in FIG. 4B.

Returning to FIGS. 1-3, the smartwatch 10 can also include one or more additional functionalities 34 (FIG. 2) that can also be controlled by associated Apps (not shown). One of the icons 24 can be associated with each additional functionality 34. Examples of additional functionalities include, but are not limited to, a camera; an accelerometer; a pedometer; a fitness/activity tracker; an altimeter; a barometer; a compass; a global positioning system; a sleep monitor; a fall sensor; a microphone; and a speaker.

Referring to FIGS. 1 and 2, the smartwatch 10 can also display time and/or date at a display area 36 on the touchscreen display 16. The time can be kept using a time-keeping circuit 38 known in the art and the date can be kept using a date-keeping circuit 40 known in the art. The time-keeping circuit 38 and the date-keeping circuit 40 can be circuitry that obtains time and date data from a source external to the smartwatch 10. The smartwatch 10 further includes a wireless communication system 42 for communicating with one or more external devices (discussed below with respect to FIGS. 4A and 4B). The wireless communication system 42 can be known wireless communication technologies such as Bluetooth®. In one embodiment, the wireless communication system 42 can operate using short-wavelength ultrahigh frequency radio waves from 2.402 GHz to 2.480 GHz. A data storage 44 is also provided for storing data, such as data collected by the sensors 26, 30. One or more rechargeable batteries 46 provide electrical power for powering operation of the smartwatch 10. In addition, as described further below, the smartwatch 10 may optionally further include a data analysis system 48 that analyzes data collected by the smartwatch 10.

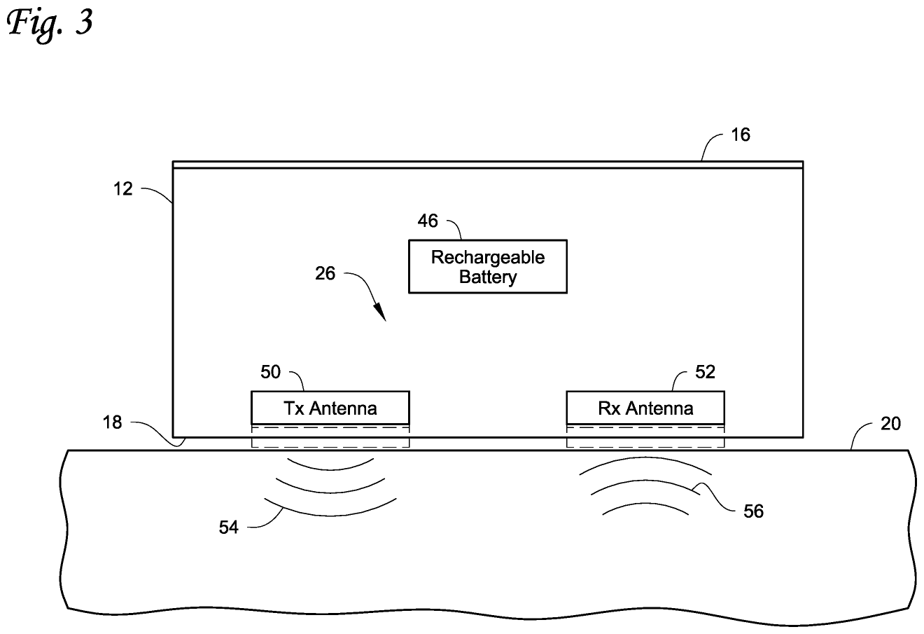

Referring to FIG. 3, an example construction of the non-invasive analyte sensor 26 is depicted. The sensor 26 is depicted as including an antenna array that includes at least one transmit antenna/element 50 (hereinafter “transmit antenna 50”) and at least one receive antenna/element 52 (hereinafter “receive antenna 52”). The transmit antenna 50 is positioned, arranged and configured to transmit a signal 54 that is the radio frequency (RF) or microwave range of the electromagnetic spectrum into the user, for example the user's arm 20. The transmit antenna 50 can be an electrode or any other suitable transmitter of electromagnetic signals in the radio frequency (RF) or microwave range. The transmit antenna 50 can have any arrangement and orientation relative to the user's arm 20 that is sufficient to allow the analyte sensing to take place. In one non-limiting embodiment, the transmit antenna 50 can be arranged to face in a direction that is substantially toward the user's arm 20.

The transmit signal can be generated by a transmit circuit (not shown) that is controlled by a controller (not shown). Transmit circuits for generating transmit signals in the RF or microwave frequency range are well known in the art. In one embodiment, the transmit circuit can include, for example, a connection to the battery 46, a frequency generator, and optionally filters, amplifiers or any other suitable elements for a circuit generating an RF or microwave frequency electromagnetic signal. In an embodiment, the signal generated by the transmit circuit can have at least two discrete frequencies (i.e. a plurality of discrete frequencies), each of which is in the range from about 10 kHz to about 100 GHz. In another embodiment, each of the at least two discrete frequencies can be in a range from about 300 MHz to about 6000 MHz. In an embodiment, the transmit circuit can be configured to sweep through a range of frequencies that are within the range of about 10 kHz to about 100 GHz, or in another embodiment a range of about 300 MHz to about 6000 MHz. In an embodiment, the transmit circuit can be configured to produce a complex transmit signal, the complex signal including a plurality of signal components, each of the signal components having a different frequency. The complex signal can be generated by blending or multiplexing multiple signals together followed by transmitting the complex signal whereby the plurality of frequencies are transmitted at the same time. Further information on transmit antennas, transmit circuits and controllers can be found in U.S. Pat. No. 10,548,503, U.S. Patent Application Publication 2019/0008422, and U.S. Patent Application Publication 2020/0187791, the entire contents of each are incorporated herein by reference in their entirety.

The receive antenna(s) 52 is positioned, arranged, and configured to detect one or more electromagnetic response signals 56 that result from the transmission of the transmit signal 54 by the transmit antenna 50 into the user and impinging on an analyte. The receive antenna 52 can be an electrode or any other suitable receiver of electromagnetic signals in the radio frequency (RF) or microwave range. In an embodiment, the receive antenna 52 is configured to detect electromagnetic signals having at least two frequencies, each of which is in the range from about 10 kHz to about 100 GHz, or in another embodiment a range from about 300 MHz to about 6000 MHz. The receive antenna 52 can have any arrangement and orientation relative to the arm 20 that is sufficient to allow detection of the response signal(s) 56 to allow the analyte sensing to take place. In one non-limiting embodiment, the receive antenna 52 can be arranged to face in a direction that is substantially toward the arm 20.

A receive circuit (not shown) is electrically connectable to the receive antenna 52 and conveys the received response from the receive antenna 52 to the controller. The receive circuit can have any configuration that is suitable for interfacing with the receive antenna 52 to convert the electromagnetic energy detected by the receive antenna 52 into one or more signals reflective of the response signal(s) 56. The construction of receive circuits are well known in the art. The receive circuit can be configured to condition the signal(s) prior to providing the signal(s) to the controller, for example through amplifying the signal(s), filtering the signal(s), or the like. Accordingly, the receive circuit may include filters, amplifiers, or any other suitable components for conditioning the signal(s) provided to the controller. In an embodiment, at least one of the receive circuit or the controller can be configured to decompose or demultiplex a complex signal, detected by the receive antenna 52, including a plurality of signal components each at different frequencies into each of the constituent signal components. In an embodiment, decomposing the complex signal can include applying a Fourier transform to the detected complex signal. However, decomposing or demultiplexing a received complex signal is optional. Instead, in an embodiment, the complex signal detected by the receive antenna can be analyzed as a whole (i.e. without demultiplexing the complex signal) to detect the analyte as long as the detected signal provides enough information to make the analyte detection. Further information on receive antennas, and receive circuits can be found in U.S. Pat. No. 10,548,503, U.S. Patent Application Publication 2019/0008422, and U.S. Patent Application Publication 2020/0187791, the entire contents of each are incorporated herein by reference in their entirety.

With continued reference to FIG. 3, the transmit antenna 50 and the receive antenna 52 can be completely disposed within the housing portion 12 (as depicted in solid lines in FIG. 3), bottoms of the transmit antenna 50 and the receive antenna 52 can be flush with and form a portion of the bottom wall of the housing portion 12, or a portion of each of the transmit antenna 50 and the receive antenna 52 can extend beyond the bottom wall and outside of the housing portion 12 (as depicted in dashed lines in FIG. 2). Further, the battery 46 can be positioned between the touchscreen display 16 and the transmit antenna 50 and the receive antenna 52.

The analyte(s) detected by the sensor 26 can be any analyte that one may wish to detect that may be present in blood or interstitial fluid in the user's arm. The analyte(s) that is detected can include, for example, naturally occurring substances, artificial substances, metabolites, and/or reaction products. For example, the analyte(s) can include, but is not limited to, one or more of blood glucose; blood alcohol; white blood cells; luteinizing hormone; insulin; acarboxyprothrombin; acylcarnitine; adenine phosphoribosyl transferase; adenosine deaminase; albumin; alpha-fetoprotein; amino acid profiles (arginine (Krebs cycle), histidine/urocanic acid, homocysteine, phenylalanine/tyrosine, tryptophan); andrenostenedione; antipyrine; arabinitol enantiomers; arginase; benzoylecgonine (cocaine); biotinidase; biopterin; c-reactive protein; carnitine; pro-BNP; BNP; troponin; carnosinase; CD4; ceruloplasmin; chenodeoxycholic acid; chloroquine; cholesterol; cholinesterase; conjugated 1-β hydroxy-cholic acid; cortisol; creatine kinase; creatine kinase MM isoenzyme; cyclosporin A; d-penicillamine; de-ethylchloroquine; dehydroepiandrosterone sulfate; DNA (acetylator polymorphism, alcohol dehydrogenase, alpha 1-antitrypsin, cystic fibrosis, Duchenne/Becker muscular dystrophy, analyte-6-phosphate dehydrogenase, hemoglobin A, hemoglobin S, hemoglobin C, hemoglobin D, hemoglobin E, hemoglobin F, D-Punjab, beta-thalassemia, hepatitis B virus, HCMV, HTLV-1, Leber hereditary optic neuropathy, MCAD, RNA, PKU, Plasmodium vivax, sexual differentiation, 21-deoxycortisol); desbutylhalofantrine; dihydropteridine reductase; diptheria/tetanus antitoxin; erythrocyte arginase; erythrocyte protoporphyrin; esterase D; fatty acids/acylglycines; free β-human chorionic gonadotropin; free erythrocyte porphyrin; free thyroxine (FT4); free tri-iodothyronine (FT3); fumarylacetoacetase; galactose/gal-1-phosphate; galactose-1-phosphate uridyltransferase; gentamicin; analyte-6-phosphate dehydrogenase; glutathione; glutathione perioxidase; glycocholic acid; glycosylated hemoglobin; halofantrine; hemoglobin variants; hexosaminidase A; human erythrocyte carbonic anhydrase I; 17-alpha-hydroxyprogesterone; hypoxanthine phosphoribosyl transferase; immunoreactive trypsin; lactate; lead; lipoproteins ((a), B/A-1, β); lysozyme; mefloquine; netilmicin; phenobarbitone; phenytoin; phytanic/pristanic acid; progesterone; prolactin; prolidase; purine nucleoside phosphorylase; quinine; reverse tri-iodothyronine (rT3); selenium; serum pancreatic lipase; sissomicin; somatomedin C; specific antibodies (adenovirus, anti-nuclear antibody, anti-zeta antibody, arbovirus, Aujeszky's disease virus, dengue virus, Dracunculus medinensis, Echinococcus granulosus, Entamoeba histolytica, enterovirus, Giardia duodenalisa, Helicobacter pylori, hepatitis B virus, herpes virus, HIV-1, IgE (atopic disease), influenza virus, Leishmania donovani, leptospira, measles/mumps/rubella, Mycobacterium leprae, Mycoplasma pneumoniae, Myoglobin, Onchocerca volvulus, parainfluenza virus, Plasmodium falciparum, polio virus, Pseudomonas aeruginosa, respiratory syncytial virus, rickettsia (scrub typhus), Schistosoma mansoni, Toxoplasma gondii, Trepenoma pallidium, Trypanosoma cruzi/rangeli, vesicular stomatis virus, Wuchereria bancrofti, yellow fever virus); specific antigens (hepatitis B virus, HIV-1); succinylacetone; sulfadoxine; theophylline; thyrotropin (TSH); thyroxine (T4); thyroxine-binding globulin; trace elements; transferrin; UDP-galactose-4-epimerase; urea; uroporphyrinogen I synthase; vitamin A; and zinc protoporphyrin.

The analyte(s) can also include one or more chemicals introduced into the user. The analyte(s) can include a marker such as a contrast agent, a radioisotope, or other chemical agent. The analyte(s) can include a fluorocarbon-based synthetic blood. The analyte(s) can include a drug or pharmaceutical composition, with non-limiting examples including ethanol; cannabis (marijuana, tetrahydrocannabinol, hashish); inhalants (nitrous oxide, amyl nitrite, butyl nitrite, chlorohydrocarbons, hydrocarbons); cocaine (crack cocaine); stimulants (amphetamines, methamphetamines, Ritalin, Cylert, Preludin, Didrex, PreState, Voranil, Sandrex, Plegine); depressants (barbiturates, methaqualone, tranquilizers such as Valium, Librium, Miltown, Serax, Equanil, Tranxene); hallucinogens (phencyclidine, lysergic acid, mescaline, peyote, psilocybin); narcotics (heroin, codeine, morphine, opium, meperidine, Percocet, Percodan, Tussionex, Fentanyl, Darvon, Talwin, Lomotil); designer drugs (analogs of fentanyl, meperidine, amphetamines, methamphetamines, and phencyclidine, for example, Ecstasy); anabolic steroids; and nicotine. The analyte(s) can include other drugs or pharmaceutical compositions. The analyte(s) can include neurochemicals or other chemicals generated within the body, such as, for example, ascorbic acid, uric acid, dopamine, noradrenaline, 3-methoxytyramine (3MT), 3,4-Dihydroxyphenylacetic acid (DOPAC), Homovanillic acid (HVA), 5-Hydroxytryptamine (5HT), and 5-Hydroxyindoleacetic acid (FHIAA).

With reference to FIG. 4A, a system 60 that includes the smartwatch 10 is depicted. The system 60 includes a mobile device 62 and a server 64. The mobile device 62 can be a mobile phone, a tablet, a laptop, or a device specifically constructed for communication with the smartwatch 10. The smartwatch 10 and the mobile device 62 are in wireless (or wired) communication with one another, for example via Bluetooth® or other wireless communication technology. The communication between the smartwatch 10 and the mobile device 62 permits data to flow therebetween. For example, data collected by the smartwatch 10 can be transmitted to the mobile device 62. In some embodiments, the data analysis system 48 can be provided on the mobile device 62 for analyzing data collected by and received from the smartwatch 10. Data can also be transmitted from the mobile device 62 to the smartwatch 10, for example results of the data analysis and/or healthcare suggestions or instructions to the user that are developed based on the data analysis and that are displayed on the touchscreen display or audibly presented to the user via a speaker of the smartwatch 10.

With continued reference to FIG. 4A, the server 64 can be in wireless (or wired) communication with the smartwatch 10 either directly or indirectly via the mobile device 62. The communication between the server 64 and the smartwatch 10 and/or the mobile device 62 permits data to flow therebetween. For example, data collected by the smartwatch 10 can be transmitted to the server 64 directly or indirectly via the mobile device 62. In some embodiments, the data analysis system 48 can be provided on the server 64 for analyzing data collected by and received from the smartwatch 10. Data can also be transmitted from the server 64 to the smartwatch 10, for example results of the data analysis and/or healthcare suggestions or instructions to the user that are developed based on the data analysis and that are displayed on the touchscreen display or audibly presented to the user via a speaker of the smartwatch 10.

FIG. 4B illustrates another embodiment of the system 60 where the second sensor 30 is separate from the smartwatch 10. The second sensor 30 can be a non-wearable sensor or a wearable sensor. The second sensor 30 may be in direct communication with the smartwatch 10. Analyte-related data from the smartwatch 10 and the data collected by the second sensor 30 can be transmitted to the mobile device 62 for analysis, or relayed to the server 64 by the mobile device 62 for analysis. Alternatively, the data from the smartwatch 10 and the second sensor 30 can be transmitted directly to the server 64.

The data analysis system 48, whether it is in the smartwatch 10, the mobile device 62 or the server 64, receives data relating to an analyte detected by the non-invasive analyte sensor of the smartwatch 10 and receives additional data collected by the smartwatch or by a separate sensor. The additional data can be data collected by the second sensor 30 (FIG. 2 and FIG. 4B), data relating to time or data, and/or data resulting from one or more of the additional functionality 34 (FIG. 2). The data analysis system 48 is programmed to analyze the received data and determine one or more correlations between the analyte-related data and the additional data.

In some embodiments, the analyte-related data and the additional data can be one-time readings taken at a particular time. In other embodiments, the analyte-related data and the additional data can be gathered over a sensing period. The sensing period can be any time period over which one may be able to determine a correlation between the analyte-related data collected by the smartwatch 10 and the additional data collected by the smartwatch 10. For example, the sensing period may be about equal to or greater than 1 hour, equal to or greater than 8 hours, equal to or greater than 24 hours, equal to or greater than 1 week, equal to or greater than 1 month, equal to or greater than 1 year, or shorter or longer sensing periods.

Correlations between the analyte-related data collected by the smartwatch 10 and the additional data can include, but are not limited to, determining changes in the analyte level (i.e. amount of analyte) relative to the additional data so that the wearer of the smartwatch may alter (increase or decrease) their analyte level, or determining lack of changes in the analyte level relative to the additional data so that the wearer of the smartwatch may maintain their analyte level. For example, if one assumes the analyte being detected by the analyte sensor of the smartwatch is blood glucose and the additional data is the heart rate of the user, the data analysis system 48 may determine that there is a correlation, based on the data provided to the data analysis system 48, between the blood glucose level and the heart rate including, but not limited to, the blood glucose level going down as the user's heart rate goes up. Based on this determined correlation, a signal can be sent to the smartwatch 10 (and/or to the user's mobile device 62) advising the user of this correlation and/or indicating to the user that the user should exercise more in order to reduce his or her blood glucose level. Alternatively, the data analysis system 48 could determine that the user's blood glucose level remains at a particular value over a range of heart rates in which case a signal can be sent to the smartwatch 10 (and/or to the user's mobile device 62) advising the user of this correlation and/or indicating to the user that the user should keep his or her heart rate in this range in order to maintain his or her blood glucose level.

Another example of a possible correlation between the analyte-related data and the additional data includes, but is not limited to, a correlation between blood glucose levels detected by the analyte sensor of the smartwatch and blood pressure levels. In this example, the data could indicate that the user's blood glucose increases as their blood pressure increases. Based on this determined correlation, a signal can be sent to the smartwatch 10 (and/or to the user's mobile device 62) advising the user of this correlation and/or indicating to the user that the user should take steps to reduce his or her blood pressure reduce his or her blood glucose level. Many other correlations between an analyte and other data collected by the smartwatch and optionally the separate second sensor 30 are possible.

In some embodiments, the analyte sensor of the smartwatch can be used to detect two or more analytes in the user, and the data can be analyzed to look for a correlation between the two analytes. For example, assuming the analyte sensor detects both blood glucose levels and blood alcohol levels, the data collected by the smartwatch may reveal a decrease or an increase in blood glucose levels with rising blood alcohol levels.

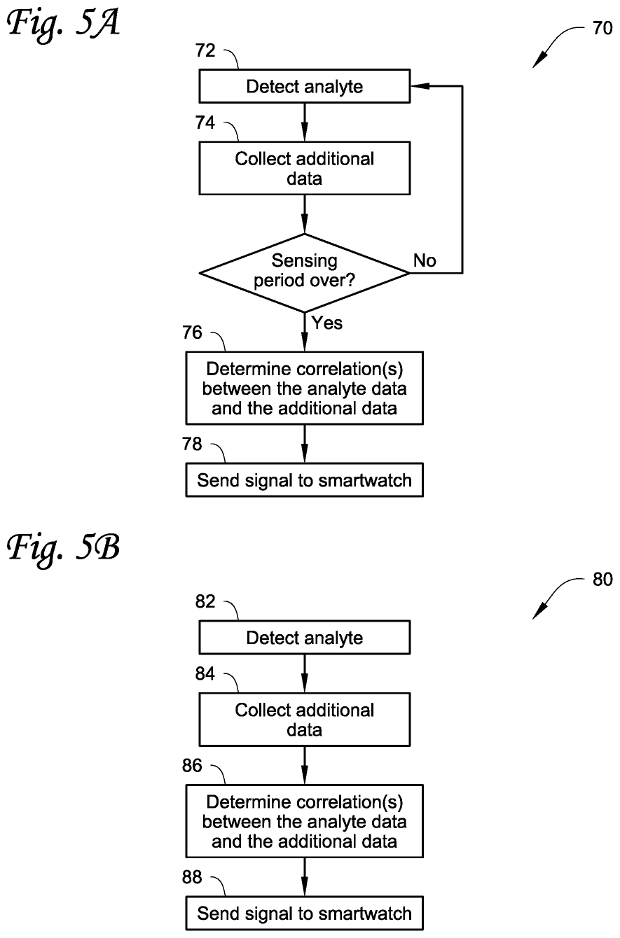

Referring to FIG. 5A, an example of a method 70 that can be implemented using the smartwatch described herein is illustrated. Assuming the smartwatch is being worn by the user, the method 70 includes a step 72 of using the non-invasive analyte sensor on the smartwatch to detect and collect analyte data regarding at least one analyte in the user. In addition, in a step 74, additional data is collected by the smartwatch (or by the separate sensor). The steps 72, 74 preferably occur substantially contemporaneously and in this embodiment occur over a sensing period. The collected data may be gathered and stored, and transmitted from the smartwatch at the end of the sensing period or at selected times during the sensing period. Or the collected data may be transmitted from the smartwatch as it is collected and stored in the mobile device 62 or stored in the server 64 for later analysis or ongoing analysis. If the sensing period is not over, the process returns to steps 72 and 74 to continue collecting more data. If the sensing period is over, in step 76 the data is analyzed by the data analysis system and any correlations between the analyte data and the additional data are determined.

A signal(s) can be generated based on the results of the analysis, and in step 78, the signal(s) can be sent to the smartwatch, either directly or via the user's mobile device. The signal(s) can cause the smartwatch and/or the mobile device to generate at least one signal that provides an indication to the user regarding the results of the data analysis. The signal generated by the smartwatch and/or the mobile device can be a visual signal (for example, a message, a numerical value, or a graphical display on the touchscreen display) and/or audible alarm or message. The signal(s) sent to the smartwatch can also include instructions to the user on steps to take to regulate the analyte, such as blood glucose.

FIG. 5B illustrates another example of a method 80 that can be implemented using the smartwatch described herein is illustrated. In the method 80, data is not collected over a sensing period. Instead, data readings are taken at a moment in time, and the data is analyzed to determine a correlation. Assuming the smartwatch is being worn by the user, the method 80 includes a step 82 of using the non-invasive analyte sensor on the smartwatch to detect and collect analyte data regarding at least one analyte in the user. In addition, in a step 84, the additional data is collected, for example by the smartwatch or by the separate sensor. The data is sent to the data analysis system, and in step 86 the data is analyzed by the data analysis system and any correlations between the analyte data and the additional data are determined. A signal(s) can be generated based on the results of the analysis, and in step 88, the signal(s) can be sent to the smartwatch, either directly or via the user's mobile device.

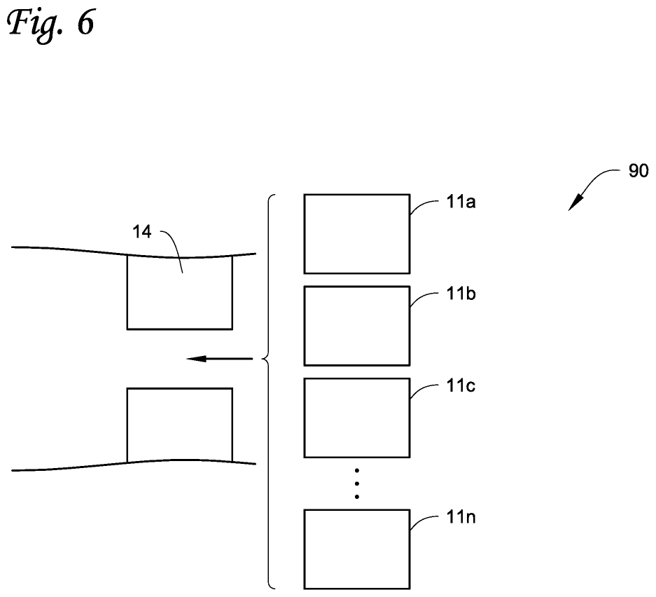

FIG. 6 illustrates an embodiment of an interchangeable smartwatch system 90. In this embodiment, a plurality of separate sensing devices 11a, 11b, 11c, . . . 11n can be interchangeably mounted on the wristband 14. Each sensing device 11a, 11b, 11c, . . . 11n can be similar to the sensing device 11 in FIGS. 1-3 including having a non-invasive analyte sensor 26. However, the sensing capability of each sensing device 11a, 11b, 11c, . . . 11n can be different from one another. For example, one of the sensing devices 11a, 11b, 11c, . . . 11n can be specifically configured to detect glucose, another one of the sensing devices 11a, 11b, 11c, . . . 11n can be specifically configured to detect alcohol, etc. Depending upon the sensing needs of the user, the user can change the sensing device 11a, 11b, 11c, 11n to install a sensing device 11a, 11b, 11c, . . . 11n with a desired sensing function.

The examples disclosed in this application are to be considered in all respects as illustrative and not limitative. The scope of the invention is indicated by the appended claims rather than by the foregoing description; and all changes which come within the meaning and range of equivalency of the claims are intended to be embraced therein.

1. A smartwatch, comprising:
a housing portion having a touchscreen display on an upper face thereof;
at least one rechargeable battery within the housing portion providing electrical power;
a non-invasive analyte sensor at least partially in the housing portion, the non-invasive analyte sensor includes:
at least one transmit antenna and at least one receive antenna, the at least one transmit antenna is positioned and arranged to transmit a signal into a person wearing the smartwatch, wherein the signal is in a radio or microwave frequency range of the electromagnetic spectrum, and the at least one receive antenna is positioned and arranged to detect a response resulting from transmission of the signal by the at least one transmit antenna into the person;
a first stored mobile application that interfaces with and controls operation of the non-invasive analyte sensor and whereby an analyte measurement obtained by the non-invasive analyte sensor can be displayed on the touchscreen display;
a second sensor that is configured to sense a variable physiological property of the person;
a time-keeping circuit whereby a current time can be displayed on the touchscreen display;
a date-keeping circuit whereby a current date can be displayed on the touchscreen display;
a wristband attached to the housing portion.

2. The smartwatch of claim 1, further comprising:
a second stored mobile application that interfaces with and controls operation of the second sensor and whereby a variable physiological property of the person obtained by the second sensor can be displayed on the touchscreen display.

3. The smartwatch of claim 2, wherein the second sensor comprises one of the following:
a thermometer; a heart rate monitor; a blood pressure monitor; an oxygen saturation monitor; a bioelectric impedance monitor.

4. The smartwatch of claim 1, further comprising a wireless communication mechanism.

5. The smartwatch of claim 1, wherein the at least one transmit antenna and the at least one receive antenna are positioned adjacent to a bottom face of the housing portion opposite the touchscreen display.

6. The smartwatch of claim 1, wherein the battery is positioned between the at least one transmit antenna and the at least one receive antenna and the touchscreen display.

7. The smartwatch of claim 3, further comprising at least one of the following: a camera; an accelerometer; a pedometer; a fitness/activity tracker; an altimeter; a barometer; a compass; a global positioning system; a sleep monitor; a fall sensor; a microphone; and a speaker.

8. A system, comprising:
the smartwatch of claim 1;
a data analysis system in electronic communication with and receiving data collected by the smartwatch, the data including first data on an analyte detected by the non-invasive analyte sensor and second data collected by the second sensor, and the data analysis system is programmed to determine a correlation between the first data and the second data.

9. The system of claim 8, wherein the data analysis system is part of the smartwatch.

10. The system of claim 8, wherein the data analysis system is separate from the smartwatch.

11. An interchangeable smartwatch system, comprising:
a wristband having a mounting portion;
a plurality of sensing devices, each sensing device is configured to be removably mountable to the mounting portion of the wristband, and the sensing devices have different sensing capabilities from one another;
each sensing device having:
a housing portion having a touchscreen display on an upper face thereof;
at least one rechargeable battery within the housing portion providing electrical power;
a non-invasive analyte sensor at least partially in the housing portion, the non-invasive analyte sensor includes:
at least one transmit antenna and at least one receive antenna, the at least one transmit antenna is positioned and arranged to transmit a signal into a person wearing the smartwatch, wherein the signal is in a radio or microwave frequency range of the electromagnetic spectrum, and the at least one receive antenna is positioned and arranged to detect a response resulting from transmission of the signal by the at least one transmit antenna into the person;
a first stored mobile application that interfaces with and controls operation of the non-invasive analyte sensor and whereby an analyte measurement obtained by the non-invasive analyte sensor can be displayed on the touchscreen display.

12. The interchangeable smartwatch system of claim 11, wherein one of the sensing devices is configured to sense glucose, and one of the sensing devices is configured to sense an analyte other than glucose.

13. The interchangeable smartwatch system of claim 11, wherein each one of the sensing devices comprises a second sensor that is configured to sense a variable physiological property of the person.

14. The interchangeable smartwatch system of claim 13, wherein the second sensor comprises one of the following:
a thermometer; a heart rate monitor; a blood pressure monitor; an oxygen saturation monitor; a bioelectric impedance monitor.

15. A method comprising:
using a non-invasive analyte sensor on a smartwatch worn by a person to collect analyte data by sensing an analyte in the person, the non-invasive analyte sensor includes at least one transmit antenna and at least one receive antenna, the at least one transmit antenna is positioned and arranged to transmit a signal into the person wearing the smartwatch, wherein the signal is in a radio or microwave frequency range of the electromagnetic spectrum, and the at least one receive antenna is positioned and arranged to detect a response resulting from transmission of the signal by the at least one transmit antenna into the person;
collecting additional data by the smartwatch or a sensing device separate from the smartwatch; and
sending the collected analyte data and the additional data to a data analysis system.

16. The method of claim 15, further comprising:
the data analysis system using the collected analyte data and the additional data to determine a correlation between the analyte and the additional data.

17. The method of claim 16, further comprising:
based on the determined correlation, sending a signal to the smartwatch instructing the person to engage in an activity that causes a change in the level of the analyte.
